(19) United States
(12) Patent Application Publication
(10) Pub. No.: US 2006/0134007 A1
(43) Pub. Date: Jun. 22, 2006

(54) Blister for inhalers

(75) Inventors: Michael Krueger, Ingelheim (DE); Burkhard Metzger, Ingelheim (DE); Michael Trunk, Ingelheim (DE); Joerg Schiewe, Mainz (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/253,312

(22) Filed: Oct. 19, 2005

(30) Foreign Application Priority Data
Oct. 21, 2004 (EP) 04025038

(51) Int. Cl. A61K 9/14; A61M 11/00; A61L 9/04

(52) U.S. Cl. 424/46; 128/200.23

(57) ABSTRACT

The invention relates to a blister for charging an inhaler with a pharmaceutical preparation, characterised in that the blister is at least partially manufactured from a dehydrating plastics material. The invention also relates to blisters according to the invention which are filled with a pharmaceutical preparation.

The invention relates to a primary packaging for inhalation formulations as an integral component of a serviceable powder inhaler, in particular for storing pharmaceutical composition formulations which contain tiotropium bromide monohydrate.

### PRIOR ART

The medical aerosol therapy oriented toward pulmonary inhalation by means of nebulisers, metered-dose aerosols or dry powder inhalers plays an important role in the treatment of numerous lung diseases.

Single-dose and multi-dose apparatuses are known in the field of powder inhalers. In single-dose powder inhalers the dosing can be undertaken in the form of capsules which contain a powder formulation. Powder formulations contain the active ingredient in micronised form (with a particle size of approx 1 to 5 μm), and generally one or more auxiliary agent(s). If a capsule is used as a container then this is opened in the powder inhaler, prior to the inhalation manoeuvre, by piercing, crushing or cutting, so the powder can be conveyed from the capsule by the patient's breath and an airborne aerosol is produced which the patient inhales. A distinction is made in powder inhalers between multi-dose powder inhalers which contain the formulation in the form of a powder supply from which the respective single dose is removed by an incorporated dosing unit, and powder inhalers with pre-dosed, packed single doses which are either stored together in the apparatus or are inserted individually into the apparatus on use.

Within the scope of the present invention multi-dose powder inhalers with pre-dosed, packed single doses which are arranged in a blister strip or in a blister disc are of particular interest. In any case the manner in which the powder formulation is packed in the apparatus is decisive for the product quality and thus for the suitability for inhalative applications.

Examples of inhalers which build on the said two principles are known in the prior art. Thus DE 3348370 and DE 3336486 disclose inhalers which contain a disc-shaped blister packaging comprising a plurality of circularly arranged cavities. The individual cavities each contain a dose of a drug powder intended for inhalation. The cavities are sealed on both sides, for example by a sealing foil. The cavity is opened to deploy the drug powder. An air duct connects the opened cavity to the mouthpiece of the inhaler. The inhaler of DE 3336486 is described in more detail by way of example. This comprises a housing in which there is located a chamber (supply chamber) comprising an air inlet and in which a disc-shaped round blister with packaged drug pockets is located. The blister is loosely connected to a round rotatable disc. Peripheral holes, which are in contact with the drug pockets in the axial direction, are formed on the disc, i.e. the pockets and holes are located above and below each other. The chamber comprises an air outlet. The inhaler also comprises a plunger which is arranged such that it can pierce open a respective drug pocket, so the drug is released into the chamber and can be inhaled via a mouthpiece. Reference is made to the drawings of the patent application and the US patent specification.

DE 4106379 describes an inhaler into which a blister or the like for a powdery drug can be introduced. The blister comprises two material webs that can be separated from each other and which define at least one container in which the drug is located. The apparatus is provided with a device which, for opening a container, separates the two material webs from each other at an opening station. The user can inhale the powdery drug from the opened container via an outlet part, for instance a mouthpiece which is connected to the opened container. In the process one of the material webs can also be a carrier web with a plurality of pockets and the other material web a covering web. Each pocket and the adjoining region of the covering web then form a container. A driving device, which separates the carrier web and the covering web from each other, can be provided at the opening station. This driving device comprises, for example two driving wheels (for example toothed wheels), which between them hold the covering web in driving engagement. Each individual blister defines a type of supply chamber, which is connected via an air duct to the mouthpiece, in the inhaler in this case as well.

A distinction is made between the primary packaging and the secondary packaging with respect to the packaging of the drug powder.

The primary packaging is characterised in that it is situated in direct contact with the inhalation formulation.

The primary packaging can optionally be surrounded by a second outer form of protection, the secondary packaging means.

The primary packaging means can in this case be for example a capsule, a rigid or flexible blister with cavities or a disc with cavities. Capsules are excluded within the scope of the present invention, however.

The secondary packaging means can be a blister, a pocket, a bag, or a different type of container. The secondary packaging means usually completely surrounds the primary packaging means. Secondary packaging means are used in particular if the primary packaging means does not provide sufficient protection against moisture.

The primary packagings can be manufactured from synthetic plastics materials, such as polyethylene, polycarbonate, polyester, polypropylene or polyethylene terephthalate inter alia. Composite materials made of plastics material and aluminium or just aluminium, for example, are also suitable.

The primary packaging means and optionally the secondary packaging means have the function of protecting the active ingredient as well as the entire inhalation formulation from chemical or physical change. Physical changes include, in particular, changes which can change the deployment of the predetermined fine particle dose. The term “fine particle dose” is in this case taken to mean the dose which reaches the patient's lungs. It is influenced by the interactions of the micronised active ingredient particles among themselves and the interactions with the auxiliaries. It has been found that these interactions can be changed, in particular by a change in the degree of moisture in the interior of the packaging, in such a way that the fine particle dose is considerably changed, in particular is much reduced. Changes of this type include the penetration of water into the packaging as well as the removal of water from the interior of the packaging.

A primary function of the packaging therefore is to keep the chemical composition of the atmosphere in the interior of the packaging constant in order to prevent physical or chemical changes in the active ingredient formulation, and to keep the inhalation formulation stable. In this connection a distinction is made between a stability oriented toward a short time, which the inhalation formulation must possess per se even if it is not adequately protected by the packaging means (“in-use stability”) and the long-term stability, i.e. the stability which has to be ensured as long as the inhalation formulation is in the unopened packaging means.

The packaging must therefore ensure that the inhalation formulation remains stable in the long term. If the material and the constructional configuration of the primary packaging means cannot ensure this then a secondary packaging means is required. The choice of a suitable material for the primary packaging means is determined by two factors: on the one hand, the material must be able to fulfil the protective function discussed and, on the other hand, the material must be such that the primary packaging can be given the form necessary for use in the powder inhaler and the primary packaging means can fulfil the function intended for it.

Plastics materials from the group comprising thermoplastic polymers, such as polystyrenes, polyolefins, polyamides, polyvinylchlorides or polyurethanes, are preferably used. These have the necessary rigidity and mobility to fulfil the mechanical functions of the primary packaging means. Their drawback is that they are permeable to atmospheric humidity in both directions. There is thus a need to increase the capacity for the packagings to stably store the inhalation powder.

Surprisingly it has accordingly been observed that pharmaceutical formulations which contain tiotropium bromide monohydrate as the active ingredient react in terms of their physico-chemical properties to a change in the degree of moisture. A solution to this problem could be found in that materials in which a dehydrating agent is incorporated were used for the primary packaging means.

### DESCRIPTION OF THE INVENTION

The present invention therefore relates to packagings in the form of a blister strip or a blister disc for inhalation powder containing a moisture-sensitive pharmaceutical composition or a moisture-sensitive formulation for use as an integral component of a serviceable powder inhaler in order to protect this pharmaceutical composition or formulation from absorbing moisture (for example absorbing water).

A further object of the packaging consists in minimising the exchange of substance between its interior and the environment.

The container preferably consists of a plastics material into which a dehydrating material is incorporated and which fundamentally improves the chemical and physical stability of the pharmaceutical preparation.

A further object of the invention consists in packing inhalation formulations containing a moisture-sensitive pharmaceutical composition in a multi-dose powder inhaler with pre-dosed single doses such that the penetration of moisture into the formulation is delayed compared with the containers known from the prior art.

### DESCRIPTION OF THE INVENTION IN DETAIL

The invention relates to a blister selected from the group comprising blister strips or blister discs for packing inhalation formulations containing a moisture-sensitive pharmaceutical composition or a moisture-sensitive pharmaceutical composition formulation, the blister firstly comprising a base element which consists of a thermoplastic polymer and at least two cavities separated from each other by a web. The cavities are open at least toward one side, optionally also to two opposing sides. These openings are closed in the serviceable blister, for example by a sealing foil rigidly connected to the base element.

Blister discs are preferred.

The inhalation powder is situated in the cavities. The filled blister with closed cavities is an integral component of a serviceable powder inhaler.

The blister is characterised in that at least a portion of the wall of the base element consists of a dehydrating material.

The configuration of the blister according to the invention is stipulated by the powder inhaler used. The base element can be configured in such a way that the pharmaceutical formulation is directly or indirectly in contact therewith.

With the blister according to the invention, an inhalation formulation, which preferably contains a moisture-sensitive anticholinergic, optionally in an amorphous state, and which has to be stored in a powder inhaler for a relatively long time before it is deployed, is better protected from the penetration of moisture from the external environment than is the case with comparable blisters known from the prior art.

The reduced penetration of moisture into the blisters improves the long-term stability and the stability after opening of an inhalation formulation.

The long-term stability is measured in a packaged and sealed blister or a packaged and sealed powder inhaler by long-term instances of storage in which the extension to the moisture protection is illustrated as a function of the blister or powder inhaler leakage rate in mg/year by the percentage by weight of molecular screen.

The stability after opening is measured in a powder inhaler that is no longer packaged and sealed (i.e. opened) or the capsule removed from a blister unit. The measuring parameters correspond to the details as illustrated for measuring the long-term stability.

The long-term stability and stability after opening are measured, for example, at 30 to 40° C. and 75% humidity in blister packagings or powder inhalers filled and not filled with inhalation powder.

According to the invention it is not necessary, but preferred, for all walls of the cavity to consist of the same material. At least the wall sealing the opening can be made of a different material to the remaining walls in a cavity.

“Inhalation formulation” in this case preferably represents a pharmaceutical powder formulation which contains an anticholinergic as the active component and of which the particles are less than 100 micrometers in size.

“Integral component of a serviceable inhaler” means that the blister is an element in the inhaler without which the charging of the inhaler with the pharmaceutical composition formulation (inhalation formulation) for the purpose of inhalation is not possible or is not provided. This element can be rigidly connected to the inhaler in the ready-to-use state (serviceable state), so it cannot be removed without destruction or without damaging the inhaler, or it is loosely connected to the inhaler in a destruction-free manner or detachably connected thereto.

“Serviceable” means that the container according to the invention is inserted into the inhaler. The container is optionally opened mechanically by components of the inhaler and/or transported in the inhaler to the site of deployment. The sequence of these two steps can also be changed.

The material for the base element is preferably a polymer composition which contains at least one thermoplastic polymer, at least one dehydrating agent and optionally at least one elastomer and/or optionally plasticisers and/or further fibres. The material contains nether gelatine, nor cellulose or starches or derivatives thereof.

Preferred polymer compositions for example comprise:
50 to 80% by weight of one or more thermoplastic polymer(s)
b) 20 to 50% by weight of one or more dehydrating agent(s)
2 to 8% by weight of one or more elastomer(s)
Optionally 1 to 4% by weight of a synthetic and/or vegetable and/or animal fibre 0.5 to 4 mm in length.

The quantity of dehydrating agent is preferably below 30% by weight, more preferably up to 25% by weight.

Thermoplastic polymers, such as polystyrenes, polyolefins, polyamides, polyvinylchlorides or polyurethanes are primarily considered as the polymer components of the plastics material. Polyethylenes are preferred, in particular polyethylene with a density between 900 and 1,000 kg/m³, preferably from 940 to 980 kg/m³, particularly preferably of 960 kg/m³ (high-density polyethylene), polycarbonate, polyester, polypropylene or polyethylene terephthalate.

Silica gels, activated carbons, zeolites, aluminium oxide, magnesium sulphate, molecular screens inter alia are examples of possible dehydrating agents.

As elastomers one or more substance(s) can be provided, for example, from the group comprising styrene butadiene rubbers (SBR), styrene ethylene butylene styrene copolymers (SEBS), butyl rubbers, ethylene propylene rubbers (EPR), ethylene propylene diene rubbers (EPDM), ethylene vinylacetate copolymers (EVA), ethylene acrylate copolymers, acrylonitrile butadiene copolymers, polynorbonenes, polyisoprenes, polychloroprenes and polybutadienes.

Finally, the polymer composition can also contain further inorganic or organic additives which have the following function: plasticiser, stabiliser, dye, pigment or the like.

Dehydrating plastics materials, i.e. plastics materials which contain a dehydrating agent, which can be injection moulded or blow moulded are preferably used. In addition, plastics materials are preferred, for the processing of which no mould release agent, which can bring about adhesion of the charge to the wall, is required. This has the advantage that the interior of the container does not have to be cleaned of mould release agent in order, for example, to satisfy the official regulations (for example to DAB (German Pharmacopoeia)) which restrict the use of mould release agents for primary packaging means.

In a preferred embodiment the dehydrating plastics material does not have any pronounced adhesion to pharmaceutical-chemical substances, in particular to particles of respirable size, so when using the blister in an inhaler the entire contents of the cavity can be released. This ensures more exact dosing, in particular of the respirable fine fraction of the pharmaceutical preparation.

Further details relating to the composition or processing can be found in the prior art, in particular EP599690, EP432438 or EP400460.

In one embodiment the blister wall can contain regions with a composition that is different to that of polymer/dehydrating agent. It is thus possible to configure the regions of the container at which the inhaler-integral means act to open the cavity from a different material than the remainder of the wall. The dehydrating agents can also be used only in regions which do not come into contact with the inhalation formulation, etc.

In other embodiments the wall of the cavity comprises at least two layers, an inner layer and at least one outer layer located thereabove. In this case the inner layer forms the direct wall of the cavity and is thus in contact with the inhalation formulation. In this case the inner layer preferably consists of a polymer composition without dehydrating agent. The second layer, surrounding this first layer, which does not come into contact with the inhalation formulation consists of the polymer composition with dehydrating agent. In this case the polymers of the inner and the at least one second layer can be selected such that they are not connected to each other with integral fit or that they are connected to each other with integral fit.

In yet a further embodiment the base element is manufactured from a material without dehydrating agent onto which, after filling and closing of the cavities, a layer of polymer composition with dehydrating agent is applied.

The blister according to the invention primarily offers advantages when active ingredients, auxiliaries or formulations have to be protected in particular from absorbing water. This applies by way of example to inhalation powders which were produced by means of spray drying and/or to active ingredients, auxiliaries and formulations which exist in the amorphous state.

Active ingredients that are effective by inhalation are preferably used.

Particularly preferred in this connection are pharmaceutical compositions which are selecting from the group comprising anticholinergics, betamimetics, steroids, phosphodiesterase IV-inhibitors, LTD4 antagonists and EGFR kinase inhibitors, triptanes, CGRP antagonists, phosphodiesterase V-inhibitors and combinations of two or more such active ingredients, for example a betamimetic plus an anticholinergic or the combination of two or more pharmaceutical compositions from the same group, for example the combination of two or more anticholinergics.

In particular the following examples are cited for the effective components or the salts thereof:

Anticholinergics used are preferably selected from the group comprising tiotropium or tiotropium bromide, oxitropium bromide, flutropium bromide, ipratropium bromide, glycopyrronium salts, trospium chloride, tolterodine, 2,2-diphenylpropionic acid tropenol ester methobromide, 2,2-diphenylpropionic acid scopine ester methobromide, 2-fluoro-2,2-diphenyl acetic acid scopine ester methobromide, 2-fluoro-2,2-diphenyl acetic acid tropenol ester methobromide, 3,3′,4,4′-tetrafluorobenzillic acid tropenol ester methobromide, 3,3′,4,4′-tetrafluorobenzillic acid scopine ester methobromide, 4,4′-difluorobenzillic acid tropenol ester methobromide, 4,4′-difluorobenzillic acid scopine ester methobromide, 3,3′-difluorobenzillic acid tropenol ester methobromide, 3,3′-difluorobenzillic acid scopine ester methobromide, 9-hydroxy-fluorene-9-carboxylic acid tropenol ester methobromide, 9-fluoro-fluorene-9-carboxylic acid tropenol ester methobromide, 9-hydroxy-fluorene-9-carboxylic acid scopine ester methobromide, 9-fluoro-fluorene-9-carboxylic acid scopine ester methobromide, 9-methyl-fluorene-9-carboxylic acid tropenol ester methobromide, 9-methyl-fluorene-9-carboxylic acid scopine ester methobromide, benzillic acid cyclopropyltropine ester methobromide, 2,2-diphenylpropionic acid cyclopropyltropine ester methobromide, 9-hydroxy-xanthene-9-carboxylic acid cyclopropyltropine ester methobromide, 9-methyl-fluorene-9-carboxylic acid cyclopropyltropine ester methobromide, 9-methyl-xanthene-9-carboxylic acid cyclopropyltropine ester methobromide, 9-hydroxy-fluorene-9-carboxylic acid cyclopropyltropine ester methobromide, 4,4′-difluorobenzillic acid methylester cyclopropyltropine ester methobromide, 9-hydroxy-xanthene-9-carboxylic acid tropenol ester methobromide, 9-hydroxy-xanthene-9-carboxylic acid scopine ester methobromide, 9-methyl-xanthene-9-carboxylic acid tropenol ester methobromide, 9-methyl-xanthene-9-carboxylic acid scopine ester methobromide, 9-ethyl-xanthene-9-carboxylic acid tropenol ester methobromide, 9-difluormethyl-xanthene-9-carboxylic acid tropenol ester methobromide and 9-hydroxymethyl-xanthene-9-carboxylic acid scopine ester methobromide, optionally in the form of their racemates, enantiomers or diastereomers and optionally in the form of their acid addition salts, solvates and/or hydrates.

Betamimetics used are preferably selected from the group comprising albuterol, bambuterol, bitolterol, broxaterol, carbuterol, clenbuterol, fenoterol, formoterol, hexoprenaline, ibuterol, indacterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramides, terbutaline, tolubuterol, CHF-1035, HOKU-81, KUL-1248, 3-(4-{6-[2-hydroxy-2-(4-hydroxy-3-hydroxymethyl-phenyl)-ethylamino]-hexyloxy)-butyl)-benzenesulphonamide, 5-[2-(5,6-diethyl-indan-2-ylamino)-1-hydroxy-ethyl]-8-hydroxy-1H-quinolin-2-one, 4-hydroxy-7-[2-{[2-{[3-(2-phenylethoxy)propyl]-sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol and 1-(4-ethoxycarbonylamino-3-cyano-5-fluorophenyl)-2-(tert.-butylamino)ethanol, optionally in the form of their racemates, enantiomers or diastereomers and optionally in the form of their pharmacologically acceptable acid addition salts, solvates and/or hydrates.

Steroids used are preferably selected from the group comprising prednisolone, prednisone, butixocortpropionate, RPR-106541, flunisolide, beclomethasone, triamcinolone, budesonide, fluticasone, mometasone, ciclesonide, rofleponide, ST-126, dexamethasone, 6α,9α-difluoro-17α-[(2-furanylcarbonyl)oxy]-11β-hydroxy-16α-methyl-3-oxo-androsta-1,4-diene-17β-carbothionic acid (S)-fluoromethylester, 6α,9α-difluoro-11β-hydroxy-16α-methyl-3-oxo-17α-propionyloxy-androsta-1,4-diene-17β-carbothionic acid (S)-(2-oxo-tetrahydro-furan-3S-yl)ester and etiprednol-dichloroacetate (BNP-166), optionally in the form of their racemates, enantiomers or diastereomers and optionally in the form of their salts and derivatives, their solvates and/or hydrates.

PDE IV-inhibitors used are preferably selected from the group comprising enprofylline, theophylline, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofylline, atizoram, V-11294A, C1-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of their racemates, enantiomers or diastereomers and optionally in the form of their pharmacologically acceptable acid addition salts, solvates and/or hydrates.

LTD4-antagonists used are preferably selected from the group comprising montelukast, 1-(((R)-(3-(2-(6,7-difluoro-2-quinolinyl)ethenyl)phenyl)-3-(2-(2-hydroxy-2-propyl)phenyl)thio)methylcyclopropane acetic acid, 1-(((1(R)-3(3-(2-(2,3-dichlorothieno[3,2-b]pyridin-5-yI)-(E)-ethenyl)phenyl)-3-(2-(1-hydroxy-1-methylethyl)phenyl)propyl)thio)methyl)cyclopropane acetic acid, pranlukast, zafirlukast, [2-[[2-(4-tert-butyl-2-thiazolyl)-5-benzofuranyl]oxymethyl]phenyl]acetic acid, MCC-847 (ZD-3523), MN-001, MEN-91507 (LM-1507), VUF-5078, VUF-K-8707 and L-733321, optionally in the form of their racemates, enantiomers or diastereomers, optionally in the form of their pharmacologically acceptable acid addition salts, and optionally in the form of their salts and derivatives, their solvates and/or hydrates.

EGFR kinase inhibitors used are selected from the group comprising cetuximab, trastuzumab, ABX-EGF, Mab ICR-62, 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1phenyl-ethyl)amino]-6-{[4-(morpholin-4-yl)-1-oxo-2-buten-1-yl]amino}-7-cyclopentyloxy-quinazoline, 4-[(3-chloro-4-fluorophenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluorophenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethinyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethinyl-phenyl)amino]-6-{[4-(5,5-dimethyl-2-oxo-morpholin-4-yl)-1-oxo-2-buten-1-yl]amino}-quinazoline, 4-[(3-chloro-4-fluorophenyl)amino]-6-{2-[4-(2-oxo-morpholin-4-yl)-piperidin-1-yl]-ethoxy}-7-methoxy-quinazoline, 4-[(3-chloro-4-fluorophenyl)amino]-6-(trans-4-amino-cyclohexan-1-yloxy)-7-methoxy-quinazoline, 4-[(3-chloro-4-fluorophenyl)amino]-6-(trans-4-methanesulphonylamino-cyclohexan-1-yloxy)-7-methoxy-quinazoline, 4-[(3-chloro-4-fluorophenyl)amino]-6-(tetrahydropyran-3-yloxy)-7-methoxy-quinazoline, 4-[(3-chloro-4-fluorophenyl)amino]-6-{1-[(morpholin-4-yl)carbonyl]-piperidin-4-yloxy}-7-methoxy-quinazoline, 4-[(3-chloro-4-fluorophenyl)amino]-6-(piperidin-3-yloxy)-7-methoxy-quinazoline, 4-[(3-chloro-4-fluorophenyl)amino]-6-[1-(2-acetylamino-ethyl)-piperidin-4-yloxy]-7-methoxy-quinazoline, 4-[(3-chloro-4-fluorophenyl)amino]-6-(tetrahydropyran-4-yloxy)-7-ethoxy-quinazoline, 4-[(3-chloro-4-fluorophenyl)amino]-6-{trans-4-[(morpholin-4-yl)carbonylamino]-cyclohexan-1-yloxy}-7-methoxy-quinazoline, 4-[(3-chloro-4-fluorophenyl)amino]-6-{1-[(piperidin-1-yl)carbonyl]-piperidin-4-yloxy}-7-methoxy-quinazoline, 4-[(3-chloro-4-fluorophenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluorophenyl)amino]-6-(trans-4-methanesulphonylamino-cyclohexan-1-yloxy)-7-methoxy-quinazoline, 4-[(3-chloro-4-fluorophenyl)amino]-6-(1-methanesulphonyl-piperidin-4-yloxy)-7-(2-methoxy-ethoxy)-quinazoline, 4-[(3-chloro-4-fluorophenyl)amino]-6-[1-(2-methoxy-acetyl)-piperidin-4-yloxy]-7-(2-methoxy-ethoxy)-quinazoline, 4-[(3-ethinyl-phenyl)amino]-6-(tetrahydropyran-4-yloxy]-7-methoxy-quinazoline, 4-[(3-chloro-4-fluorophenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluorophenyl)amino]-6-{cis-4-[(morpholin-4-yl)carbonylamino]-cyclohexan-1-yloxy}-7-methoxy-quinazoline, 4-[(3-chloro-4-fluorophenyl)amino]-6-{1-[2-(2-oxopyrrolidin-1-yl)ethyl]-piperidin-4-yloxy}-7-methoxy-quinazoline, 4-[(3-ethinyl-phenyl)amino]-6-(1-acetyl-piperidin-4-yloxy)-7-methoxy-quinazoline, 4-[(3-ethinyl-phenyl)amino]-6-(1-methyl-piperidin-4-yloxy)-7-methoxy-quinazoline, 4-[(3-ethinyl-phenyl)amino]-6-(1-methanesulphonyl-piperidin-4-yloxy)-7-methoxy-quinazoline, 4-[(3-chloro-4-fluorophenyl)amino]-6-(1-methyl-piperidin-4-yloxy)-7(2-methoxy-ethoxy)-quinazoline, 4-[(3-ethinyl-phenyl)amino]-6-{1-[(morpholin-4-yl)carbonyl]-piperidin-4-yloxy}-7-methoxy-quinazoline, 4-[(3-chloro-4-fluorophenyl)amino]-6-{1-[(N-methyl-N-2-methoxyethyl-amino)carbonyl]-piperidin-4-yloxy}-7-methoxy-quinazoline, 4-[(3-chloro-4-fluorophenyl)amino]-6-(1-ethyl-piperidin-4-yloxy)-7-methoxy-quinazoline, 4-[(3-chloro-4-fluorophenyl)amino]-6-[cis-4-(N-methane sulphonyl-N-methyl-amino)-cyclohexan-1-yloxy]-7-methoxy-quinazoline, 4-[(3-chloro-4-fluorophenyl)amino]-6-[cis-4-(N-acetyl-N-methyl-amino)-cyclohexan-1-yloxy]-7-methoxy-quinazoline, 4-[(3-chloro-4-fluorophenyl)amino]-6-(trans-4-methylamino-cyclohexan-1-yloxy)-7-methoxy-quinazoline, 4-[(3-chloro-4-fluorophenyl)amino]-6-[trans-4-(N-methane sulphonyl-N-methyl-amino)-cyclohexan-1-yloxy]-7-methoxy-quinazoline, 4-[(3-chloro-4-fluorophenyl)amino]-6-(trans-4-dimethylamino-cyclohexan-1-yloxy)-7-methoxy-quinazoline, 4-[(3-chloro-4-fluorophenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluorophenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluorophenyl)amino]-6-(1-methanesulphonyl-piperidin-4-yloxy)-7-methoxy-quinazoline, 4-[(3-chloro-4-fluorophenyl)amino]-6-(1-cyano-piperidin-4-yloxy)-7-methoxy-quinazoline, and 4-[(3-chloro-4-fluorophenyl)amino]-6-{1-[(2-methoxyethyl)carbonyl]-piperidin-4-yloxy)-7-methoxy-quinazoline, optionally in the form of their racemates, enantiomers or diastereomers, optionally in the form of their pharmacologically acceptable acid addition salts, their solvates and/or hydrates.

Pharmaceutical compositions with the above-mentioned active ingredients are considered, as well as their salts, esters and the combination of these active ingredients, salts and esters, for inhalation.

The inhalation powders are produced according to the method known from the prior art, but in particular by spray drying methods.

The inhalation powders used in the blisters according to the invention preferably contain at least one auxiliary agent in addition to the active ingredient. This can comprise an auxiliary agent fraction that is uniform with respect to the mean particle size of the auxiliary agent particle (for example 15 to 80 micrometers) or optionally be a mixture of coarser auxiliary agent with a mean particle size of 15 to 80 micrometers and finer auxiliary agent with a mean particle size of 1 to 9 micrometers. If auxiliary agent mixtures of coarser and finer auxiliary agent fractions are used, the fraction of finer auxiliary agent in the total auxiliary agent quantity is preferably 1 to 20%.

Monosaccharides (for example glucose or arabinose), disaccharides (for example lactose, saccharose, maltose), oligo- and polysaccharides (for example dextranes), polyalcohols (for example sorbitol, mannitol, xylitol), salts (for example sodium chloride, calcium carbonate) or mixtures of these auxiliaries are cited as examples of physiologically safe auxiliaries. Within the context of the invention lactose is particularly preferably used and lactose monohydrate is very particularly preferably used as the auxiliary.

The previously described blisters are usually primary packagings which can exist in the form of a cylindrical disc or a strip-shaped elastic blister. As described below the blisters can be opened to release the inhalation formulation either with a piercing element or by separating the material webs from each other. The blisters can be equipped with one or more single doses. These can additionally be equipped with a polymer and/or aluminium layer. The serviceable inhaler can also be stored welded in a bag made of a polymer or an aluminium foil. The use of a secondary packaging means has various advantages. On the one hand, the functionality of the primary packagings according to the invention, in particular the functionality of the dehydrating agent, is extended in terms of time in that the time-limited barrier effect against water is only effective after opening of the secondary packaging, and this provides additional security of the product quality. On the other hand, the storage time moisture protection and moisture protection during open storage in the unopened secondary packaging is extended in that moisture penetrating into the secondary packaging is absorbed by the dehydrating plastics material.

The blister disc according to the invention can be a cylindrical disc up to 5 mm in height and with a diameter of up to 15 cm. Troughs or holes (cavities) are formed in the disc perpendicular to the disc plane. The troughs and holes are preferably formed on the outer edge of this disc and can be sealed by one or more foil(s). The inhalation formulation is situated in the troughs or holes. The disc body consists of the material used according to the invention. A disc of this type can, for example, be used in an inhaler according to DE 3348370 or DE 3336486. An inhaler of this type comprises a housing in which the disc-shaped round blister with packed drug pockets is situated. The inhaler comprises inter alia a pin which is arranged such that in each case it can open a drug pocket, so the drug is released into the chamber and can be inhaled via a mouthpiece.

In a further embodiment the container is a strip-shaped elastic blister with drug pockets, as is described in DE 4106379. The blister comprises at least two material webs that can be separated from each other, which define at least two containers in which the drug is situated in each case. The material can in this case be the material described at the outset as being suitable for the invention. In variants of this embodiment the primary packaging is a comparable blister in which an aluminium foil and a foil according to the material of the invention are laminated. In this case the side coming into contact with the inhalation formulation can be the aluminium side as well as the polymer side provided with the dehydrating agent. In yet further variants these are laminates of the layer sequence: aluminium, polymer with dehydrating agent, aluminium. The associated inhaler is provided with a device which, for opening this blister, separates the two material webs from each other at an opening station. The user can inhale the powdery drug from the opened container via an outlet part, for instance a mouthpiece, which is connected to the opened container. One of the material webs can also be a carrier web with a plurality of pockets and the other material web a covering web. Each pocket and the adjoining region of the covering web then form a container. A driving device, which separates the carrier web and the covering web from each other, can be provided at the opening station. This driving device comprises, for example two driving wheels (for example toothed wheels), which between them hold the covering web in driving engagement. Each individual blister defines a type of supply chamber, which is connected via an air duct to the mouthpiece, in the inhaler in this case as well.

In an alternative according to the invention bodies made of the polymer material, described at the outset, with dehydrating properties are admixed with the inhalation formulations. The size of these mould parts is greater than the largest particle of the pharmaceutical composition formulation, so the bodies can be separated from the formulation particles by screens. So these bodies are not inhaled as well they have to be screened out during inhalation. For this purpose, the mouthpieces or other regions of the inhaler can be provided with a corresponding screen. In other embodiments the bodies are securely connected in the interior of the cavity of the container to at least one of the container walls. In yet another embodiment the bodies are constructed in such a way that they are too heavy to be carried along with the pharmaceutical composition particles during the inhalation process. The dehydrating plastics material can be introduced into the capsule in the form of one or more body/bodies. When using more than one body identical or different geometries can in each case be used in one capsule. Spherical or ellipsoid of revolution-shaped geometries are preferred.

In this case the base part of the blister does not have to be manufactured from the polymer containing the dehydrating agent. In an embodiment with a freely moving mould part made of a material containing a dehydrating agent, the formulation and the mould part can exist in a two-part telescopic capsule which can be inserted into appropriate inhalers. Capsules of this type are described, for example in EP 1100474 to which reference is made in its entirety. Preferred capsule size is 3. A suitable inhaler is, for example, an apparatus with the HandiHaler® trade mark, as is disclosed for example in EP 1342483. A preferred embodiment of this aspect of the invention relates to an ensemble of an inhaler for the inhalation of powdery pharmaceutical compositions and a two-part capsule which contains the mould part according to the invention, the inhaler being characterised by a) a beaker-like lower part that is open toward the top and that comprises two opposing windows in the casing and has a first hinge element at the edge of the opening, b) a plate which covers the opening of the lower part and comprises a second hinge element, c) an inhalation chamber for receiving the capsule which is formed perpendicular to the plate plane at the side of the plate pointing toward the lower part and on which is provided a head that can move toward a spring, the head being provided with two sharpened needles, d) an upper part with a mouth pipe and a third hinge element and e) a cover which comprises a fourth hinge element, the hinge elements—one of the lower part, two of the plate, three of the upper part and four of the cover—being connected to each other.

### EXAMPLE 1

The extension of the protection of blister packs against penetrating moisture in long-term and open storage at 40° C. and 75% r.h. (containers filled with inhalation powder comprising a tiotropium/lactose mixture):

Using suitable measuring methods it was ascertained that blister packs in long-term and open storage at 40° C. and 75% r.h. (r.h.=relative humidity) let water through the blisters at a rate of 0.1 to 100 mg per year. The quantity of water absorbed depends very much on the blister materials used and the tightness of the seal. The following table contains an overview which shows the extension to the moisture protection as a function of the blister leakage rate in mg/year and of the percentage by weight of molecular screen in % by weight.

| | | | | | | Extension to | |
|---|---|---|---|---|---|---|---|
| Total mass MS* | | Water absorption | | Blister leakage | | moisture | |
| in the blister | | capacity MS* | | rate per year | | protection | |
| 10 | mg | 1.5 | mg | 0.1 | mg | +15.0 | years |
| 39 | mg | 5.9 | mg | 1 | mg | +5.9 | years |
| 65 | mg | 9.8 | mg | 5 | mg | +2.0 | years |
| 117 | mg | 17.6 | mg | 10 | mg | +1.8 | years |
| 78 | mg | 11.7 | mg | 10 | mg | +1.2 | years |
| 130 | mg | 19.5 | mg | 20 | mg | +1.0 | years |
| 260 | mg | 30.0 | mg | 50 | mg | +0.8 | years |
| 195 | mg | 29.3 | mg | 50 | mg | +0.6 | years |
| 390 | mg | 58.5 | mg | 100 | mg | +0.6 | years |

*MS: molecular screen

The values in the “Extension to moisture protection” column show that with low blister leakage rates of 1 mg/year or lower an extension to the protection of 6 years or more can be attained under ambient conditions of 40° C. and 75% r.h. With high blister leakage rates of 100 mg water per year an extension to the moisture protection by 7 months is attained under the same ambient conditions. This example can be directly transferred to the situation of stability after opening of the blister pack if the described blister pack is in a hermetically sealed external packaging for long-term storage.

1. A blister which is an integral component of a serviceable powder inhaler, selected from blister strips or blister discs, comprising a base element with at least two cavities that are open toward at least one side and are separated from each other by a web and which contain a moisture-sensitive inhalation formulation, and the where at least one opening of the cavities is sealed, wherein at least a portion of the wall of the base element consists of a dehydrating material.

2. The blister according to claim 1, wherein the dehydrating material is a polymer composition comprising one or more different polymer(s) and/or one or more different dehydrating agents.

3. The blister according to claim 1 or claim 2, wherein the dehydrating material comprises silica gels, activated carbon, zeolites, aluminium oxide, magnesium sulphate and molecular screens.

4. The blister according to claim 2, wherein the polymers used are thermoplastic polymers, selected from polystyrenes, polyolefins, polyamides, polyvinylchlorides or polyurethanes, polyethylene, polycarbonate, polyester, polypropylene or polyethyleneterephthalate.

5. The blister according to claim 2, wherein the polymer composition also contains one or more elastomers, selected from the group consisting of styrene butadiene rubbers, styrene ethylene butylene styrene copolymers, butyl rubbers, ethylene propylene rubbers, ethylene propylene diene rubbers, ethylene vinylacetate copolymers, ethylene acrylate copolymers, acrylonitrile butadiene copolymers, polynorbonenes, polyisoprenes, polychloroprenes and polybutadienes.

6. The blister according to claim 2, wherein the polymer composition contains plasticisers, stabilisers, dyes or pigments.

7. The blister according to claim 1, wherein the inhalation formulation contains tiotropium bromide monohydrate.

8. The blister according to claim 1, wherein, as an additional element, the blister contains a body which consists of dehydrating material.

9. The blister according to claim 8, wherein the dehydrating material of the additional element is a polymer composition comprising one or more different polymer(s) and/or one or more dehydrating agents.

10. The blister according to claim 1 or claim 8, wherein the blister is a blister disc.

11. the blister according to claim 1 or claim 8, wherein the blister is a disc-shaped blister up to 5 mm high and with a diameter of up to 15 cm, on the outer edge of which and perpendicular to the disc plane troughs or holes are formed, of which the openings are closed by one or more foil(s).

12. The blister according to claim 1 or claim 8, wherein the blister is a pierceable blister.

13. The blister according to claim 1 or claim 8, wherein the blister is a strip-shaped elastic blister with drug pockets and comprises at least two material webs that can be separated from each other.

14. A container comprising the blister according to claim 13, wherein the material webs comprise a laminate made of aluminium and a dehydrating material.

15. An inhaler containing a blister according to claim 1, wherein the inhaler can contain one or more single dose(s).

16. A pocket containing at least one active ingredient suitable for inhalation and at least one auxiliary suitable for inhalation and at least one mould part that is movable in the pocket and made of a dehydrating material.

17. The pocket according to claim 16, wherein, as an additional element, the pocket contains a body which consists of dehydrating material.

18. The pocket according to claim 17, wherein the dehydrating material of the additional element is a polymer composition comprising one or more different polymer(s) and/or one or more different dehydrating agents.
